(19) United States
(12) Patent Application Publication
(10) Pub. No.: US 2014/0331601 A1
(43) Pub. Date: Nov. 13, 2014

(54) THERMOPLASTIC COMPOSITIONS THAT INCLUDE SORBENT PARTICLES, AND METHODS OF USING THE SAME

(71) Applicant: H.B. Fuller Company, St. Paul, MN (US)

(72) Inventors: Steven A. Jorissen, Vadnais Heights, MN (US); Steven R. Vaughan, Chisago lake Township, MN (US); Sharf U. Ahmed, Woodbury, MN (US); Hassan H. Rmaile, Stillwater, MN (US); David B. Malcolm, Maplewood, MN (US); Anne E. Spinks, Hugo, MN (US); Kevin P. Davis, North St. Paul, MN (US)

(73) Assignee: H.B. Fuller Company, St. Paul, MN (US)

(21) Appl. No.: 14/264,915

(22) Filed: Apr. 29, 2014

Related U.S. Application Data
(60) Provisional application No. 61/821,599, filed on May 9, 2013.

(51) Int. Cl. C09J 11/04; B65B 55/00; C09J 143/00; A61L 9/014; C09J 153/02

(52) U.S. Cl. CPC C09J 11/04; A61L 9/014; C09J 153/02; C09J 143/00; B65B 55/00; A61L 2209/22
USPC 53/400; 523/102

(57) ABSTRACT

A thermoplastic composition is disclosed that includes a thermoplastic polymer and volatile organic compound sorbent particles, and that exhibits a viscosity of no greater than 15,000 centipoise at 177° C.

### CROSS-REFERENCE TO RELATED APPLICATIONS

This application claims the benefit of U.S. Provisional Application No. 61/821,599, filed May 9, 2013, and incorporated herein.

### BACKGROUND

The invention relates to the sorption of volatile organic compounds.

Some consumers are aware of the “new car smell” that is present in the interior of many new cars. The new car smell could be attributed to a number of factors including, e.g., any one of the many components that are within the car interior including leather, chemicals used to treat the leather, plastic components, fabrics, carpeting, cleaners, and the adhesives that are used to bond together various component of the car.

Many articles that are smaller than cars are made with components that include polymers, oils, plasticizers, surfactants, fragrances, masking agents, adhesives, and films. These components have the potential to emit a perceptible odor. In addition, the articles are often packaged in packaging materials that emit their own odor. Some of the packaging materials that are used to package such articles are airtight and do not breathe, i.e., do not allow air from the inside of the package to escape or air from the outside of the package to enter. Other packaging materials, although perforated, have insufficient airflow to permit a sufficient amount of an enclosed odor from escaping. As a result, the odors that are emitted by the articles and packaging become trapped within the interior of the package. When a user subsequently opens the package, the odors are emitted and the user may find them to be unexpected and undesirable.

There is a need to remove or reduce the presence of unexpected and undesirable odors from manufactured goods.

### SUMMARY

In one aspect, the invention features a hot melt adhesive composition that includes from about 5% by weight to about 80% by weight thermoplastic polymer, from about 20% by weight to about 70% by weight tackifying agent, and from about 0.5% by weight to about 10% by weight volatile organic compound sorbent particles, the composition exhibiting a viscosity of no greater than 15,000 centipoise (cP) at 177° C. In some embodiments, the hot melt adhesive composition is sprayable. In another embodiment, the hot melt adhesive composition emits at least 20% less volatiles relative to the composition without of the volatile organic compound sorbent particles. In other embodiments, the hot melt adhesive composition emits at least 50% less volatiles relative to the composition without of the volatile organic compound sorbent particles.

In one embodiment, the hot melt adhesive composition is free of visible settling after aging for 24 hours at 163° C. In other embodiments, the hot melt adhesive composition is free of visible settling after aging for 72 hours at 163° C.

In another embodiment, the hot melt adhesive composition further includes from about 5% by weight to about 35% by weight plasticizer.

In other embodiments, the hot melt adhesive composition includes from about 0.5% by weight to about 5% by weight of the volatile organic compound sorbent particles. In some embodiments, the sorbent particles include at least one of zeolite, alkali metal alumino-silicate, silica gel, silica-magnesia gel, silica-alumina gel, activated carbon, activated alumina, calcium oxide, and cyclodextrin

In some embodiments, the thermoplastic polymer includes a styrene block copolymer. In other embodiments, the thermoplastic polymer includes a polyolefin including at least one of an amorphous polyalphaolefin and a metallocene catalyzed polyolefin. In another embodiment, the hot melt adhesive composition includes from about 5% by weight to about 50% by weight thermoplastic polymer including at least one of block copolymer and polyolefin, from about 30% by weight to about 65% by weight tackifying agent; and from about 5% by weight to about 35% by weight plasticizer. In some embodiments, the hot melt adhesive composition further includes from about 0.5% by weight to about 5% by weight of the volatile organic compound sorbent particles.

In one embodiment, the hot melt adhesive composition is a pressure-sensitive hot melt adhesive composition.

In another aspect, the invention features a thermoplastic composition that includes from about 5% by weight to about 90% by weight a first component selected from the group consisting of thermoplastic polymer, tackifying agent and combinations thereof; and from about 5% by weight to no greater than about 70% by weight volatile organic compound sorbent particles, the composition exhibiting a viscosity no greater than 40,000 cP at 177° C. In one embodiment, the thermoplastic composition further includes from about 5% by weight to no greater than about 50% by weight plasticizer. In another embodiment, the thermoplastic composition exhibits a viscosity no greater than 15,000 cP at 177° C. In some embodiments, the thermoplastic composition is sprayable.

In other embodiments, the first component includes thermoplastic polymer and tackifying agent.

In other aspects, the invention features an elastic attachment adhesive composition that includes from about 10% by weight to about 80% by weight thermoplastic elastomer, from about 20% by weight to about 70% by weight tackifying agent; and from about 0.1% by weight to about 10% by weight volatile organic compound sorbent particles, the composition exhibiting a viscosity of no greater than 15,000 cP at 177° C. In one embodiment, the elastic attachment adhesive composition further includes an end block resin.

In another aspect, the invention features an organic volatile compound sorbent composition that includes a carrier that includes at least one of wax, petroleum jelly, grease, oil, and from about 0.5% by weight to about 10% by weight volatile organic compound sorbent particles disposed in the carrier. In one embodiment, the sorbent composition exhibits a viscosity no greater than 15,000 cP at 177° C.

In one aspect, the invention features an elastic article that includes an elastic component, and from about 0.5% by weight to about 10% by weight volatile organic compound sorbent particles, the volatile organic compound sorbent particles being at least one of disposed in the elastic component and disposed on the elastic component.

In other aspects, the invention features a disposable absorbent article that includes an absorbent core, and a thermoplastic composition disclosed herein. In one embodiment, disposable absorbent article is sealed in a packaging material.

In other aspects, the invention features a method of reducing an odor from a package, the odor being due to volatile organic compounds, the method including surrounding an article that includes a hot melt adhesive composition or a thermoplastic composition disclosed herein with a packaging material that includes a flexible film to form the package, at least one of the article and the packaging material emitting volatile organic compounds in the absence of volatile organic compound sorbent particles.

In another aspect, the invention features a method of reducing an odor that is perceived to be present when a package is opened, the method including surrounding an article that includes a hot melt adhesive composition or a thermoplastic composition disclosed herein with a packaging material to form the package, at least one of the article and the packaging material emitting volatile organic compounds, and allowing the volatile organic compound sorbent particles to adsorb at least some of the volatile organic compounds.

In another aspect, the invention features a method of reducing an odor that is perceived to be present when a package that includes an article surrounded by a packaging material is opened, the method including adding a thermoplastic composition that includes a thermoplastic polymer and volatile organic compound sorbent particles to at least one of the article and the packaging material, and enclosing the article within the packaging material, the packaging material comprising a flexible polymer film.

In another aspect, the invention features a method of reducing the volatile organic compounds emitted by a hot melt adhesive composition, the method including combining from about 0.5% by weight to about 10% by weight volatile organic compound sorbent particles, from about 5% by weight to about 80% by weight thermoplastic polymer, from about 20% by weight to about 70% by weight tackifying agent, and plasticizer to form a hot melt adhesive composition, the hot melt adhesive composition exhibiting a viscosity of no greater than 15,000 cP at 177° C. In one embodiment, the hot melt adhesive composition emits at least 20% less organic volatile compounds relative to the composition without the volatile organic compound sorbent particles. In another embodiment, the package is an airtight, sealed package.

The invention features compositions that can reduce the amount of volatile organic compounds that are present either in the composition, itself, or in the environment in which the composition is located.

Other features and advantages will be apparent from the following description of the preferred embodiments and from the claims.

### GLOSSARY

In reference to the invention, the following term has the meaning set forth below.

The term “(meth)acrylate” refers to acrylate and methacrylate.

### DETAILED DESCRIPTION

The thermoplastic composition includes a sorbent that is capable of adsorbing or absorbing volatile organic compounds and a thermoplastic polymer. The thermoplastic composition is incorporated into articles or packaging materials to adsorb or absorb volatile organic compounds from the thermoplastic composition, itself, the environment surrounding the thermoplastic composition, or a combination thereof. The thermoplastic composition can be any one of a variety of thermoplastic compositions including, e.g., hot melt adhesive compositions (e.g., construction adhesives and elastic attachment adhesive compositions), volatile organic compound sorbent compositions, and combinations thereof. The thermoplastic composition optionally is formulated to exhibit pressure-sensitive adhesive properties.

Hot Melt Adhesive Composition

In one embodiment, the thermoplastic composition is a hot melt adhesive composition that includes a thermoplastic polymer and volatile organic compound sorbent particles. The hot melt adhesive composition exhibits a viscosity no greater than 15,000 centipoise at 177° C., no greater than 10,000 cP at 177° C., or even no greater than 10,000 cP at 150° C. The hot melt adhesive composition optionally is sprayable. One useful method of determining sprayability is the Sprayability Test Method set forth in the Example section below.

The hot melt adhesive composition preferably emits at least 20% less, at least 30% less, or even at least 50% less, volatile organic compounds relative to the composition without the adsorbent particles. One useful method of measuring the relative percentage of organic volatile compounds that are emitted from a thermoplastic composition involves a headspace measurement as set forth herein in the Test Method for Determining the Relative Amount of Volatiles Emitted by a Sample.

The hot melt adhesive composition preferably is free from visible settling after sitting for 24 hours at 163° C., or even after sitting for 72 hours at 163° C.

The hot melt adhesive composition preferably includes at least about 5% by weight, from about 5% by weight to at least about 80% by weight, from about 5% by weight to about 70% by weight, from about 5% by weight to about 50% by weight, or even from about 5% by weight to about 30% by weight, thermoplastic polymer,

from about 0.1% by weight to about 10% by weight, at least about 0.5% by weight, no greater than about 5% by weight, no greater than about 3% by weight, no greater than about 2.5% by weight, no greater than about 2% by weight, no greater than about 1.5% by weight, from about 0.1% by weight to about 7.5% by weight, or even from about 0.1 to about 6% by weight volatile organic compound sorbent particles,

at least about 20% by weight, at least about 30% by weight, from about 20% by weight to about 70% by weight, from about 30% by weight to about 65% by weight, from about 35% by weight to about 65% by weight, or even from about 50% by weight to about 70% by weight tackifying agent, and

at least about 5% by weight, from about 5% by weight to about 35% by weight, from about 5% by weight to about 30% by weight, or even from about 10% by weight to about 30% by weight plasticizer.

Thermoplastic Polymer

Thermoplastic polymers suitable for use in the hot melt adhesive composition include thermoplastic elastomers, non-elastomeric thermoplastic polymers, and combinations thereof. Useful thermoplastic elastomers include, e.g., styrene block copolymers, polyolefins (e.g., amorphous polyalphaolefins, metallocene-catalyzed polyolefins, and copolymers of ethylene and non-alphaolefin monomer), acrylic elastomers, polyurethane elastomers, and functionalized versions thereof (e.g., maleic acid and maleic anhydride functionalized), and combinations thereof. Useful non-elastomeric thermoplastic polymers include, e.g., polyolefins, alkylacrylates, and combinations thereof.

Styrene Block Copolymers

Styrene block copolymers include an aromatic vinyl polymer block and a conjugated diene polymer block, a hydrogenated conjugated diene polymer block, or a combination thereof The blocks can be arranged in a variety of configurations including, e.g., linear, branched, radial, star block, and combinations thereof. The aromatic vinyl polymer block can be derived from a variety of aromatic vinyl compounds including, e.g., styrene, alpha-methylstyrene, beta-methylstyrene, o-, p-methylstyrene, t-butyl styrene, 2,4,6-trimethylstyrene, monofluorostyrene, difluorostyrene, monochlorostyrene, dichlorostyrene, methoxystyrene, 1,3-vinylnaphthalene, vinylanthracene, indene, acenaphthylene, and combinations thereof. The diene polymer block can be derived from a variety of diene-containing compounds including, e.g., isoprene, butadiene, hexadiene, 2,3-dimethyl-1,3-butadiene, 1,3-pentadiene, and hydrogenated versions thereof, and combinations thereof.

Useful styrene block copolymers include, e.g., diblock, triblock and multiblock copolymers including, e.g., styrene-butadiene, styrene-butadiene-styrene, styrene-isoprene, styrene-isoprene-styrene, styrene-ethylene/butene, styrene-ethylene/butene-styrene, styrene-ethylene/propylene, styrene-ethylene/propylene-styrene, styrene-ethylene-ethylene/propylene-styrene, and combinations thereof.

Useful styrene block copolymers are commercially available under a variety of trade designations including, e.g., the KRATON D and G series of trade designations from Kraton Performance Polymers Inc. (Houston, Tex.) including, e.g., KRATON D 1163, D1124 , D1117, D1116K , D1118K, D1184K, D1124K, D1126K, D 1701M, and KRATON G 1652 and 1726, EUROPRENE Sol T trade designation from EniChem (Houston, Tex.), SEPTON trade designation from Septon Company of America (Pasadena, Tex.) including SEPTON S 1001 styrene-ethylene-propylene-styrene block copolymer, and SEPTON 4030, 4033, 4044, 4055 and 4077 block copolymers, the VECTOR series of trade designations from Dexco Polymers, LP (Houston, Tex.) including VECTOR 4111A, 4211A and 4114A styrene-isoprene-styrene block copolymers, 6241 styrene-butadiene-styrene block copolymers and 2411 radial block copolymers, and the SOLPRENE series of trade designations from Dynasol Elastomers (Houston, Tex.) including SOLPRENE 1205 linear diblock styrene/butadiene copolymer.

Polyolefins

Useful polyolefin polymers include, e.g., homopolymers and higher order polymers (e.g., copolymers and terpolymers) derived from alpha olefin monomers. Useful alpha-olefin monomers include, e.g., mono-alpha olefins (i.e., one unsaturated double bond), and higher order olefins (e.g., di-olefins (e.g., 1,9-decadiene) including, e.g., C₂-C₂₀ α-olefin monomers, e.g., ethylene, propylene, isobutylene, 1-butene, 1-hexene, 4-methyl-1-pentene, 1-heptene, 1-octene, nonene, decene, dodecene, cyclopentene, cyclohexene, cyclooctene, 4-methyl-pentene-1,3-methyl pentene-1,3,5,5-trimethyl-hexene-1,5-ethyl-1-nonene, and combinations thereof Examples of useful polyolefins include, e.g., polyethylene, polypropylene, propylene-ethylene copolymers, propylene-butene copolymers, and combinations thereof.

Useful classes of polyolefins include, e.g., amorphous polyalphaolefins, metallocene polyolefins, copolymers of ethylene and non-alphaolefin monomers, and combinations thereof. Amorphous polyalphaolefin polymers have varying degrees of crystallinity and are not necessarily atactic. Amorphous polyalphaolefin polymers are typically polymerized by processes that employ Ziegler-Natta catalysts resulting in a relatively broad molecular weight distribution.

Useful commercially available amorphous polyalphaolefins are available under a variety of trade designations including, e.g., the REXTAC series of trade designations from Rextac LLC (Odessa, Texas) including, e.g., REXTAC RT2730, and RT2723 the VESTOPLAST series of trade designations from Evonik Industries AG (Germany) including, e.g., VESTOPLAST 508 and 708 propylene/ethylene/butene terpolymers, the EASTOFLEX series of trade designations from Eastman Chemical (Kingsport, Tennessee) including, e.g., EASTOFLEX E1016.

Metallocene-catalyzed polymers are prepared using a constrained geometry or single site metallocene catalyst. Useful metallocene-catalyzed polymers include, e.g., homopolymers and higher order polymers (e.g., copolymers and terpolymers) of alpha olefin monomers. Useful metallocene-catalyzed polymers are commercially available under of a variety of trade designations including, e.g., the AFFINITY series of trade designations from Dow Chemical Company (Midland, Michigan) including, e.g., AFFINITY EG 8200G polyolefin plastomer and AFFINITY GA 1900 and GA 1950 metallocene polyolefin elastomer, linear ethylene polymers are commercially available under the EXACT series of trade designations from ExxonMobil (Texas), the L-MODU series of trade designations from Idemitsu Kosan Co., Ltd. (Japan) including, e.g., L-MODU 5400, 5600, and 5901, the VISTAMAXX series of trade designations from ExxonMobil Chemical Company (Houston, Tex.) including, e.g., VISTAMAXX 6102, 6202, 3000, and 2330, the LICOCENE series of trade designations from Clariant Int'l Ltd. (Muttenz, Switzerland) including, e.g., LICOCENE PP 1602 TP and PP 2602 TP, and the VERSIFY series of trade designations from Dow Chemical Co. (Midland, Mich.) including, e.g., VERSIFY propylene-ethylene plastomers and elastomers.

Non-alphaolefin monomers suitable for the copolymers of ethylene and non-alphaolefin monomer include, e.g., unsaturated esters of monocarboxylic acids having from 1 to 8 carbon atoms and monocarboxylic acids having from 1 to 8 carbon atoms. Useful copolymers of ethylene and non-alphaolefin monomers include, e.g., ethylene vinylacetate, ethylene methyl acrylate, ethylene methyl methacrylate, ethylene ethyl acrylate, ethylene n-butyl acrylate, ethylene ethylhexyl acrylate, and combinations thereof.

Acrylic Elastomers

Useful acrylic elastomers include acrylic ester homopolymers and copolymers of an acrylic ester and at least one comonomer. Useful acrylic esters include, e.g., alkyl (meth)acrylates and alkoxyalkyl (meth)acrylates. Alkyl (meth)acrylates include, e.g., alkyl (meth)acrylates that include alkyl groups having from 1 to 8 carbon atoms (e.g., methyl (meth)acrylate, ethyl (meth)acrylate, propyl (meth)acrylate, isopropyl (meth)acrylate, n-butyl (meth)acrylate, isobutyl (meth)acrylate, pentyl (meth)acrylate, isoamyl (meth)acrylate, n-hexyl (meth)acrylate, 2-ethyl (meth)acrylate, and n-octyl (meth)acrylate). Alkoxyalkyl (meth)acrylates include, e.g., 2-methoxymethyl (meth)acrylate, 2-ethoxymethyl (meth)acrylate, 2-methoxyethyl (meth)acrylate, 2-ethoxyethyl (meth)acrylate, 2-(n-propoxy)ethyl (meth)acrylate, 2-butoxyethyl (meth)acrylate, 2-(iso-butoxy)ethyl (meth)acrylate, 3-methoxypropyl (meth)acrylate, 3-ethoxypropyl (meth)acrylate, 3-(n-propoxy)propyl (meth)acrylate, 3-(n-butoxy)propyl (meth)acrylate, and combinations thereof.

Useful comonomers from which the acrylic elastomer can be derived include, e.g., alpha-olefins, acrylonitriles, carboxylic acid vinyl esters, aromatic vinyl compounds, vinyl fatty acids, and combinations thereof. Suitable alpha-olefin comonomers include, e.g., ethylene, propylene, n-butene-1, isobutene, vinyl chloride, and vinylidene chloride. Examples of suitable carboxylic acid vinyl esters comonomers include vinyl acetate, vinyl propionate, and vinyl butyrate. Suitable aromatic vinyl compounds include, e.g., styrene, alpha-methylstyrene, chlorostyrene, and vinyltoluene.

Sorbent

The volatile organic compound sorbent particles are capable of adsorbing or absorbing volatile organic compounds present in the composition or the atmosphere surrounding the composition or article in which the composition is incorporated. The volatile organic compound sorbent particles preferably exhibit sorption of at least one of hydrocarbons, organic solvents, aromatics, organic impurities, and carbon dioxide. Useful sorbents include, e.g., organic adsorbents (e.g., cyclodextrins) and inorganic sorbents including, e.g., zeolites (e.g., chabasite, gumerinite, levynite, erinite, mordenite and analcite), molecular sieves (e.g., alkali metal alumino-silicates), silica gel, silica-magnesia gel, silica-alumina gel, activated carbon, activated alumina, calcium oxide, and combinations thereof. Suitable alkali metal alumino-silicate molecular sieves include, e.g., calcium, potassium, and sodium alkali metal alumino-silicates.

The volatile organic compound sorbent particles optionally are functionalized, complexed or a combination thereof with an organic group (e.g., organosiliceous groups), a monomer, a polymer that includes a functional group (e.g., hydroxyl, acid, acid anhydride, amine, epoxy, and isocyanate groups, and combinations thereof) capable of reacting with the sorbent particle, and combinations thereof. Such polymers include, e.g., polyether sulfones, sulfonated polyethersulfones, hydroxyl group-terminated poly(ethylene oxide)s, amino group-terminated poly(ethylene oxide)s, isocyanate group-terminated poly(ethylene oxide)s, poly(esteramide-diisocyanate)s, hydroxyl group-terminated poly(propylene oxide)s, hydroxyl group-terminated co-block-poly(ethylene oxide)-poly(propylene oxide)s, hydroxyl group-terminated tri-block-polypropylene oxide)-block-poly(ethylene oxide)-block-poly(propylene oxide)s, tri-block-poly(propylene glycol)-block-poly(ethylene glycol)-block-polypropylene glycol) bis(2-aminopropyl ether), polyether ketones, poly(ethylene imine)s, poly(amidoamine)s, poly(vinyl alcohol)s, poly(allyl amine)s, poly(vinyl amine)s, and cellulosic polymers. The functionalized or complexed sorbent particles can be further functionalized, complexed (e.g., covalently bonded or grafted) or a combination thereof with a thermoplastic polymer (e.g., the thermoplastic polymers set forth herein, poly(vinylidene fluoride-co-chlorotrifluoroethylene) backbone with polystyrene sulfonic acid) side chains), and combinations thereof).

Useful sorbent particles have pores sizes that are 5 Angstrom (A), 8 A, 10 A, 30 A, 50 A, or even from 5 A to 50 A in diameter, are in a variety of forms including, e.g., powders and beads, and exhibit a particle size of from about 0.5 microns to about 15 microns, or even from about 1 micron to about 5 microns, and blends of particles having different properties including, e.g., particle size, pore size, composition, and combinations thereof. Useful molecular sieves are available under a variety of trade designations including, e.g., the MOLSIV Adsorbent Type 13X, and Molecular Sieve Type 3A, Type 4A and Type 5A, which are all commercially available from UOP LLC (a Honeywell International company, Morristown, N.J.), PHONOSORB from W.R. Grace & Co. (Cambridge, Mass.), and ZEOFLAIR and ZEOCAT high performance zeolites from Zeochem AG (Switzerland).

Other suitable adsorbents include cyclodextrins, which are cyclic oligosaccharides that include at least six D-(+)-glucopyranose units attached by alpha-(1,4) glucosidic bonds. Useful cyclodextrins include alpha-cyclodextrins, beta-cyclodextrins, and gamma-cyclodextrins, which have six, seven and eight glucose units, respectively. Cyclodextrins that include any number of glucose units including, e.g., up to twelve glucose units, are suitable. Synthetically modified cyclodextrins are also suitable including, e.g., methyl-p-cyclodextrin and hydroxypropyl-cyclodextrin.

Useful cyclodextrins include uncomplexed (i.e., free cyclodextrins), precomplexed cyclodextrins), and complexed cyclodextrins (e.g., cyclodextrins that are complexed with a perfume). Mixtures of different types of cyclodextrins are also suitable and can be used to absorb a wider range of volatile organic compounds relative to a single type of cyclodextrin.

Useful cyclodextrins are available under a variety of trade designations including, e.g., CAVAMAX natural cyclodextrins, CAVASOL W7 HP 2-hydroxypropyl-B-cyclodextrins and CAVITRON W7 HP5 and HP7 2-hydroxypropyl-B-cyclodextrins, all of which are available from WackerChemie AG (Germany) distributed by Ashland, Inc. (Covington, Ky.).

Tackifying Agent

Suitable classes of tackifying agents include, e.g., aromatic, aliphatic and cycloaliphatic hydrocarbon resins, mixed aromatic and aliphatic modified hydrocarbon resins, aromatic modified aliphatic hydrocarbon resins, and hydrogenated versions thereof; terpenes, modified terpenes and hydrogenated versions thereof; natural rosins, modified rosins, rosin esters, and hydrogenated versions thereof; low molecular weight polylactic acid; and combinations thereof. Examples of useful natural and modified rosins include gum rosin, wood rosin, tall oil rosin, distilled rosin, hydrogenated rosin, dimerized rosin, and polymerized rosin. Examples of useful rosin esters include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin. Examples of useful polyterpene resins include hydrogenated polyterpene resins, and copolymers and terpolymers of natural terpenes (e.g. styrene-terpene, alpha-methyl styrene-terpene and vinyl toluene-terpene). Examples of useful aliphatic and cycloaliphatic petroleum hydrocarbon resins include aliphatic and cycloaliphatic petroleum hydrocarbon resins including, e.g., branched and unbranched C5 resins, C9 resins, and CIO resins, and the hydrogenated derivatives thereof.

Useful tackifying agents are commercially available under a variety of trade designations including, e.g., the ESCOREZ series of trade designations from Exxon Mobil Chemical Company (Houston, Texas) including ESCOREZ 5320, 5340, 5400, 5415, 5600, 5615, and 5690, the EASTOTAC series of trade designations from Eastman Chemical (Kingsport, Tenn.) including EASTOTAC H-100R, EASTOTAC H-100L, and EASTOTAC H130W, the WINGTACK series of trade designations from Cray Valley HSC (Exton, Pa.) including WINGTACK 86, WINGTACK EXTRA and WINGTACK 95 and the PICCOTAC series of trade designations from Eastman Chemical Company (Kingsport, Tenn.) including, e.g., PICCOTAC 8095.

Plasticizer

The adhesive composition optionally includes a plasticizer. Useful classes of plasticizers include liquid and solid plasticizers. Useful plasticizers include, e.g., naphthenic petroleum-based oils, paraffinic oils (e.g., cycloparaffin oils), mineral oils, phthalate esters, adipate esters, olefin oligomers (e.g., oligomers of polypropylene, polybutene, and hydrogenated polyisoprene), polybutenes, polyisoprene, hydrogenated polyisoprene, polybutadiene, benzoate esters, animal oil, derivatives of oils, glycerol esters of fatty acids, and combinations thereof.

Useful commercially available plasticizers include plasticizers sold under the NYFLEX series of trade designations including NYFLEX 222B from Nynas Corporation (Houston, Tex.), KAYDOL OIL from Sonneborn (Tarrytown N.Y.) PARAPOL polybutene from Exxon Mobil Chemical Company (Houston, Tex.), OPPANOL polyisobutylene from BASF (Ludwigsjhafen, Germany), BENZOFLEX 352 benzoate ester plasticizer from Velsicol Chemical Company (Chicago, Ill.), KRYSTOL 550 mineral oil from Petrochem Carless Limited (Surrey, England), and CALSOL 550 oil from Calumet Specialty Products Partners, LP (Indianapolis, Ind.).

Additional Components

The hot melt adhesive composition optionally includes additional components including, e.g., waxes, stabilizers, antioxidants, additional polymers, adhesion promoters, ultraviolet light stabilizers, rheology modifiers, biocides, corrosion inhibitors, dehydrators, colorants (e.g., pigments and dyes), fillers, surfactants, flame retardants, superabsorbent polymers (i.e., polymers capable of absorbing many times their own weight in water, e.g., superabsorbent polymer particles), nanoparticles, and combinations thereof.

Useful antioxidants include, e.g., pentaerythritol tetrakis[3,(3,5-di-tert-butyl-4-hydroxyphenyl)propionate], 2,2′-methylene bis(4-methyl-6-tert-butylphenol), phosphites including, e.g., tris-(p-nonylphenyl)-phosphite (TNPP) and bis(2,4-di-tert-butylphenyl)4,4′-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, N.J.), and ETHYL 702 4,4′-methylene bis(2,6-di-tert-butylphenol). When present, the adhesive composition preferably includes from about 0.1% by weight to about 2% by weight antioxidant.

Useful classes of optional waxes include, e.g., functionalized waxes, non-functionalized waxes, and combinations thereof, including, e.g., Fischer Tropsch waxes (e.g., oxidized Fischer-Tropsch waxes), polyolefin waxes (e.g., polypropylene waxes, polyethylene waxes, high density low molecular weight polyethylene waxes, and by-product polyethylene waxes), paraffin waxes, microcrystalline waxes, metallocene waxes, vegetable waxes, animal waxes, stearamide waxes, glycerin monostearate, sorbitan monostearate, and combinations thereof.

Useful nanoparticles include particles having a dimension of from about 1 nanometer (nm) to about 100 μm. The nanoparticles can be inorganic nanoparticles, organically modified inorganic nanoparticles, and combinations thereof. The nanoparticles can be of a variety of shapes including, e.g., spherical, elliptical, cylindrical, tubular, star, planar, rhombohedral, trigonal, conical, frustoconical, and combinations thereof.

One useful class of nanoparticles includes nanoclays. Suitable nanoclays are in the form of a powder (e.g., a dry granulation) of particles, clusters of particles, platelets, clusters of platelets, and combinations thereof. Useful nanoclays include, e.g. montmorillonite, montmorillonite/smectites, illites, chlorites, bentonite, kaolinite, hectorite, and halloysite nanoclays, and combinations thereof. Nanoclays are commercially available under a variety of trade designations including, e.g., the LAPONITE series of trade designation from BYK Additives Inc. (Gonzales, Tex.).

Useful surface-modified nanoclays include nanoclays modified with an organic compound. One useful class of organic nanoclay modifiers includes quaternary ammonium salts. Examples of quaternary ammonium salts surface modifiers include dimethyl benzyl, hydrogenated tallow quaternary ammonium, dimethyl, dihydrogenated tallow quaternary ammonium, dimethyl hydrogenated tallow, 2-ethylhexyl quaternary ammonium, methyl, tallow, bio-2-hydroxyethyl, quaternary ammonium, and combinations thereof.

Commercially available surface-modified nanoclay powders are available under the CLOISITE series of trade designations and include, e.g., CLOISITE 10A, 15A, 20A, 25A and 30B, from BYK Additives Inc. (Gonzales, Texas). The CLOISITE platelets have a first dimension of about 1 nm and a second dimension of from about 70 nm to about 150 nm according to the manufacturer.

Sorbent Thermoplastic Composition

In another embodiment, the thermoplastic composition is a sorbent thermoplastic composition that includes at least one of a thermoplastic polymer and a tackifying agent, volatile organic compound sorbent particles, and, optionally, plasticizer. The sorbent thermoplastic composition exhibits a viscosity no greater than about 40,000 centipoise at 177° C., no greater than about 30,000 centipoise at 177° C., no greater than about 15,000 cP at 177° C., or even no greater than about 10,000 cP at 150° C. and optionally is sprayable.

The sorbent composition preferably is able to adsorb or absorb volatile organic compounds from an article or an environment in which the composition is disposed. The adsorbent composition preferably emits at least 20% less, at least 30% less, or even at least 50% less, volatile organic compounds relative the composition without the sorbent particles.

The sorbent composition preferably is free from settling after sitting for 24 hours at 163° C., or even after sitting for 72 hours at 163° C.

Suitable thermoplastic polymers for inclusion in the sorbent thermoplastic composition include the thermoplastic polymers set forth above in the discussion pertaining to the hot melt adhesive composition. The sorbent thermoplastic composition includes from about 0% by weight to about 90% by weight, from about 5% by weight to about 90% by weight, from about 10% by weight to about 80% by weight, or even from about 15% by weight to about 70% by weight thermoplastic polymer.

Suitable tackifying agents for inclusion in the sorbent thermoplastic composition include the tackifying agents set forth above in the discussion pertaining to the hot melt adhesive composition. The sorbent thermoplastic composition includes from 0% by weight to about 50% by weight, from about 10% by weight to about 40% by weight, or even from about 10% by weight to about 35% by weight tackifying agent.

Suitable sorbents for inclusion in the sorbent thermoplastic composition include the volatile organic compound sorbent particles set forth above in the discussion pertaining to the hot melt adhesive composition. The sorbent thermoplastic composition also includes from about 5% by weight to about 70% by weight, from about 5% by weight to about 50% by weight, from about 10% by weight to about 50% by weight, or even from about 20% by weight to about 50% by weight volatile organic compound sorbent particles.

The sorbent thermoplastic composition optionally includes from about 0% by weight to about 70% by weight, from 1% by weight to about 50% by weight, or even from about 5% by weight to about 40% by weight plasticizer. Suitable plasticizers for inclusion in the sorbent thermoplastic composition include the plasticizers set forth above in the discussion pertaining to the hot melt adhesive composition.

The sorbent thermoplastic composition optionally includes from about 0% by weight to about 20% by weight or even from about 5% by weight to about 10% by weight wax. Suitable waxes for inclusion in the adsorbent thermoplastic composition include the waxes set forth above in the discussion pertaining to the hot melt adhesive composition.

The sorbent thermoplastic composition optionally includes at least one of the additional components set forth above in the discussion pertaining to the hot melt adhesive composition.

Elastic Attachment Adhesive Composition

In another embodiment, the thermoplastic composition is an elastic attachment adhesive composition. Useful elastic attachment adhesive compositions include from thermoplastic elastomer, tackifying agent, volatile organic compound sorbent particles, optionally plasticizer, and optionally end block resin. The elastic attachment adhesive composition exhibits a viscosity of no greater than 15,000 cP at 177° C., or even no greater than about 10,000 cP at 177° C.

Suitable thermoplastic elastomers for inclusion in the elastic attachment adhesive composition include the thermoplastic elastomers set forth above in the discussion pertaining to the hot melt adhesive composition including, e.g., styrene block copolymers, polyolefin elastomers, copolymers of ethylene and non-alphaolefin monomer, acrylate elastomers, ethylene-propylene-diene terpolymers, thermoplastic polyurethane elastomers, polyisoprene, polybutadiene, and combinations thereof. The elastic attachment adhesive composition preferably includes from about 10% by weight to about 80% by weight, from about 12% by weight to about 60% by weight, or even from about 15% by weight to about 45% by weight thermoplastic elastomer.

Suitable sorbents for inclusion in the elastic attachment adhesive composition include the volatile organic compound sorbent particles set forth above in the discussion pertaining to the hot melt adhesive composition. The elastic attachment adhesive composition preferably includes from about 0.1% by weight to about 10% by weight, at least about 0.5% by weight, no greater than about 5% by weight, no greater than about 3% by weight, no greater than about 2.5% by weight, no greater than about 2% by weight, no greater than about 1.5% by weight, from about 0.1% by weight to about 7.5% by weight, or even from about 0.1 to about 6% by weight volatile organic compound sorbent particles.

Suitable tackifying agents for inclusion in the elastic attachment adhesive composition include the tackifying agents set forth above in the discussion pertaining to the hot melt adhesive composition. The elastic attachment adhesive composition preferably includes at least about 20% by weight, at least about 30% by weight, from about 20% by weight to about 70% by weight, from about 25% by weight to about 60% by weight, or even from about 30% by weight to about 50% by weight tackifying agent.

When plasticizer is present in the elastic attachment adhesive composition, the elastic attachment adhesive composition includes from about 0% by weight to about 30% by weight, from about 5% by weight to about 25% by weight, from about 10% by weight to about 25% by weight, no greater than about 30% by weight, no greater than about 25% by weight, or even no greater than about 20% by weight plasticizer. Suitable plasticizers for inclusion in the elastic attachment adhesive composition include the plasticizers set forth above in the discussion pertaining to the hot melt adhesive composition.

Useful tackifying resins that are capable of associating with an end block of the block copolymer (i.e., “end block resins”) exhibit a melting point of at least 120° C., at least 130° C. or even at least 140° C. Specific examples of useful end block resins include pure aromatic resin, blends of C8 and C9 monomer, alpha-methyl styrene, and combinations thereof and are commercially available under a variety of trade designations including, e.g., KRISTALEX 1120, KRISTALEX 5140, PLASTOLYN 290LV, PICCOTEX 120, ENDEX 155, PICCOLASTIC D125, and SYLVARES SA140. When an end block resin is present in the elastic attachment adhesive composition, it is preferably present in an amount of from 0% by weight to about 20% by weight, from about 2% by weight to about 15% by weight, or even from about 4% by weight to about 10% by weight.

The elastic attachment adhesive composition optionally includes at least one of the additional components set forth above in the discussion pertaining to the hot melt adhesive composition.

The elastic attachment adhesive composition preferably is suitable for attaching an elastic component to a substrate (e.g., a polymer film). The elastic component can be in a variety of forms including, e.g., a strand, a film, a netted material, and combinations thereof. Examples of elastic components include polyethylene elastomers, polypropylene elastomers, polyester elastomers, ethylene-propylene-diene terpolymers, styrene-isoprene-styrene, styrene-butadiene-styrene, styrene-ethylene/butylene-styrene, styrene-ethylene/propylene-styrene, polyurethane, polyisoprene, cross-linked polybutadiene, natural rubber, and combinations thereof.

Use

The thermoplastic compositions are useful in adsorbing volatile organic compounds in the thermoplastic composition (i.e., adsorbing volatile organic compounds that are emitted by at least one component of the thermoplastic composition), and the volatile organic compound sorbent particles preferably reduce the amount volatile organic compounds emitted by the thermoplastic composition. The thermoplastic compositions also can be formulated to adsorb volatile organic compounds that are emitted by components of an article on or in which it is incorporated or with which it is packaged (e.g., packaging materials). In one use, the thermoplastic composition is included in a package (e.g., a flexible film sealed, or partially sealed, package of disposable diapers) and the thermoplastic composition reduces the intensity of the odor (as perceived by the user) that emanates from the package when the package is opened relative to a package without the thermoplastic composition. The sealed package can be air tight, or air permeable.

The thermoplastic composition can be applied to or incorporated in a variety of substrates including, e.g., films (e.g., polymer film (e.g., polyolefin, polyester, metalized polymer films, multi-layer films, and combinations thereof), release liners, porous substrates, sheets (e.g., paper and fiber sheets), woven and nonwoven webs, tape backings, fibers (e.g., polymeric fibers including, e.g., polyolefin (e.g., polyethylene and polypropylene), polyester, polyacrylonitrile, polyvinyl acetate, polyvinyl chloride, polyamide, polyacrylamide, and rayon), cellulose (e.g., wood and cotton), glass, and combinations thereof), cardboard, coated cardboard, paperboard, fiber board, virgin and recycled kraft, high and low density kraft, chipboard, treated and coated kraft and chipboard, and corrugated versions of the aforementioned, clay coated chipboard carton stock, composites, leather, substrates made from fibers (e.g., virgin fibers, recycled fibers, and combinations thereof), and combinations thereof. When the thermoplastic composition is a hot melt adhesive composition, it can be used to bond at least one of the aforementioned substrates to another substrate.

The thermoplastic composition also can be incorporated into a variety of disposable absorbent articles including, e.g., wipes, tissues, towels, components of absorbent articles including, e.g., an absorbent element, absorbent cores, impermeable layers (e.g., backsheets), tissue (e.g., wrapping tissue), acquisition layers and woven and nonwoven web layers (e.g., top sheets, absorbent tissue); products for cleaning, disinfecting and polishing (e.g., wipes, covers, filters, towels, and tissue (e.g., bath and face)), nonwoven roll goods, pillows, pads, cushions, masks, body care products, laboratory coats, coveralls; products used in the medical industry including, e.g., medical gowns (e.g., protective, surgical, examination), surgical drapes, caps, gloves, face masks, bandages, wound dressings, wipes, covers, containers, filters, disposable garments, bed pads, under pads, and adsorbent garments; products used in personal care including, e.g., diapers, diaper pants, baby wipes, training pants, absorbent underpants, swimwear, feminine care products including, e.g., sanitary napkins, wipes, menstrual pads, panty liners, and panty shields; and adult care products including wipes, pads, containers, incontinence products, and urinary shields.

The thermoplastic composition is useful in a variety of forms including, e.g., as a coating (e.g., continuous coatings and discontinuous (e.g., random, pattern (e.g., spiral), and array) coatings), bead, film (e.g., continuous and discontinuous film), sheet, fiber, filament, strand, rope, nonwoven web (e.g., a spunbond, meltblown, carded, wet-laid, hydroentangled, and combinations thereof), woven webs, and combinations thereof.

Various techniques can be used to apply the thermoplastic composition to a substrate including, e.g., slot coating, roll coating, curtain coating, brush coating, spraying (e.g., spiral spraying, random spraying and random fiberization (e.g., melt blowing)), foaming, extrusion (e.g., applying a bead, fine line extrusion, single screw extrusion, and twin screw extrusion), transfer coating, screen printing, wheel application, noncontact coating, contacting coating, gravure, engraved roller, flexographic, “on demand” application methods, and combinations thereof.

In on demand hot melt application systems (which are also referred to as “tank free” and “tankless” systems), hot melt compositions are fed in a solid state (e.g., pellets), to a relatively small heating vessel (relative to traditional hot melt applications systems that include a pot) where the hot melt composition is melted and, typically shortly thereafter, the molten liquid is applied to a substrate. In on demand systems, a relatively large quantity of hot melt composition typically does not remain in a molten state for an extended period of time. In many existing on demand systems, the volume of molten hot melt composition is no greater than about 1 liter, or even no greater than about 500 milliliters, and the hot melt composition is maintained in a molten state for a relatively brief period of time, including, e.g., less than two hours, less than one hour, or even less than 30 minutes. Suitable on demand hot melt adhesive application systems include, e.g., InvisiPac Tank-Free Hot Melt Delivery System from Graco Minnesota Inc. (Minneapolis, Minn.) and the Freedom Hot Melt Dispensing System from Nordson Corporation (Westlake, Ohio). On demand hot melt adhesive application systems are described in U.S. Patent Publication Nos. 2013-0105039, 2013-0112709, 2013-0112279, and 2014-0042182, and U.S. Pat. No. 8,201,717, and incorporated herein.

Packaging

Articles that include the thermoplastic composition can be packaged in a variety of packaging materials including, e.g., polymer films (e.g., shrink films including, e.g., monovinylarene-based shrink films, biaxially oriented polymer film, monoaxially oriented film), cardboard, coated cardboard, paperboard, fiber board, virgin and recycled kraft, high and low density kraft, chipboard, treated and coated kraft and chipboard, and corrugated versions of the aforementioned, clay coated chipboard carton stock, composites, and combinations thereof.

The invention will now be described by way of the following examples. All parts, ratios, percentages and amounts stated in the Examples are by weight unless otherwise specified.

### EXAMPLES

### Test Procedures

Test procedures used in the examples include the following. All ratios and percentages are by weight unless otherwise indicated. The procedures are conducted at room temperature (i.e., an ambient temperature of from about 20° C. to about 25° C.) unless otherwise specified.

### Viscosity Test Method

Viscosity is determined in accordance with ASTM D-3236 entitled, “Standard Test Method for Apparent viscosity of Hot Melt Adhesives and Coating Materials,” (Oct. 31, 1988), using a Brookfield Thermosel viscometer Model RVDV 2 and a number 27 spindle. The results are reported in centipoise (cP).

### Sprayability Test Method

A sample hot melt composition is heated to 163° C. and sprayed onto a treated polyethylene film in a spiral pattern using 0.018 in diameter CF spray nozzle (Nordson Corporation, Westlake, Ohio). The adhesive applicator, hose and tank are heated to 163° C., the process air is heated to 177° C., and the process air pressure is less than 20 psi. The composition is deemed sprayable if it is capable of producing a continuous overlaping spiral pattern that is at least 0.5 inches wide at a web speed of 500 feet per minute (ft/min) and a coat weight of 6.2 grams per square meter (g/m²).

### Test Method for Determining the Relative Amount of Volatiles Emitted by a Sample

The relative amounts of organic volatile compounds a compound are determined using Gas Chromatograph (e.g., an Agilent 6890N) configured with a split/splitless capillary column inlet. The detector is an Agilent 5973N quadrupole mass spectrometer. The sampling device is a Hewlett Packard HP 7694 Headspace Sampler. The gas chromatograph and mass spectrometer are controlled by a PC based software system, entitled, “Agilent Chemstation.” The gas chromatograph is operated using an RTX-VOLATILES column 30 meters long, 0.25 millimeters inner diameter and having 1 micrometer thick film (Restek Corporation, Bellafonte, Pa.).

The temperature program for each analysis consists of a starting temperature of 40° C., holding at 40° C. for 6 minutes, followed by a temperature ramp up to 250° C. at a rate of 10° C. per minute for a run time of 30 minutes, exclusive of cool down and equilibration times.

The gas chromatograph is operated in constant flow mode at 1 milliliter of helium per minute. The initial head pressure at 40° C. is 7.6 pounds per square inch (psi). The gas chromatograph inlet is operated in split mode, with a split ratio of 25 to 1.

The mass spectrometer is operated in scan mode, ranging from 15 atomic mass units (amu) to 500 amu. The mass spectrometer source temperature is set at 230° C. and the quadrupoles are set at 150° C.

The headspace autosampler oven is set at 60° C. Each sample is heated at 60° C. for 60 minutes. The headspace autosampler transfer line and sample loop are kept at 110° C. during the analysis. The headspace autosampler pressurization time is 0.25 minutes, the headspace autosampler loop fill time is 0.25 minutes, the headspace autosampler loop equilibration time is 0.25 minutes and the headspace autosampler injection time is 1.0 minute.

The molten samples are weighed into 20 mL crimp-top headspace vials and sealed. The amount of sample is between 2.0 grams (g) and 2.1 g. The weight of each sample is recorded. The samples are heated in the autosampler oven using the conditions described above. After the heating period, the headspace is sampled using the headspace autosampler. Two milliliters of the headspace is injected into the gas chromatograph. An empty vial is run before the first sample as a blank to determine the cleanliness of the system. If the blank shows evidence of contamination, the system is cleaned.

Detected peaks are identified by comparing the resulting mass spectrum to those in the commercially available Wiley library of mass spectra. The detected peaks are integrated and peak areas reported.

The relative change in volatiles is determined by assigning a value of 100% to the control composition (i.e., the composition without the sorbent particles) and comparing the results of the sample composition to the control. The result is recorded in percent of the control.

The determination of the relative change in volatiles is valid between the same composition when the samples are prepared and analyzed in the same manner.

### Settling Test Method

A 300 gram sample of composition is placed in a 400 milliliter glass beaker and heated until it is molten. The molten composition is then held at a temperature to maintain it in a molten state and the composition is observed with the naked eye to determine if any particulate present in the composition has settled toward or at the bottom of the beaker.

### Example 1

A hot melt adhesive composition is prepared by combining 25% by weight VECTOR 4114 styrene block copolymer (Dexco Polymers LP, Houston, Tex.), 20% by weight naphthenic oil, 49% by weight EASTOTAC H100L hydrocarbon resin (Eastman Chemical Co., Kingsport, Tenn.), 1% by weight IRGANOX 1010 antioxidant (BASF Corp., Florham Park, N.J.), and 5% by weight MOLSIV 13X adsorbent particles (UOP LLC, Morristown, N.J.) and heating the same to from 175° C. to 180° C., with mixing, to achieve a molten liquid.

The composition is expected to have a viscosity of 13,450 cP at 163° C.

### Example 2

A thermoplastic composition was prepared by combining 38% by weight AFFINITY GA 1900 metallocene polyolefin elastomer having a density of 0.870 g/10 min. and a melt index of 1250 g/10 min at 190 CC (The Dow Chemical Co., Midland, Mich.), 31% by weight MOLSIV 13X adsorbent particles, and 31% by weight mineral oil and heating the same to from 175° C. to 180° C., with mixing, to achieve a molten liquid.

The composition had a viscosity of 1,765 cP at 177° C.

### Example 3

An elastic attachment adhesive composition is prepared by combining 25% by weight KRATON 1162 styrene-isoprene-styrene block copolymer Kraton Performance Polymers Inc. (Houston, Tex.), 15% by weight naphthenic oil, 53% by weight hydrogenated C5 hydrocarbon tackifying resin, 5% by weight end block resin, 1% by weight antioxidant, and 1% by weight MOLSIV 13X adsorbent particles. The composition is expected to exhibit a viscosity of no greater than 15,000 cP at 177° C.

### Control 1

The composition of Control 1 was a hot melt adhesive composition that included styrene-isoprene-styrene block copolymer, tackifying agent, and plasticizer that exhibited a viscosity of 9837 cP of at 163° C.

The composition of Control 1 was tested according to the Test Method for Determining the Relative Amount of Volatiles Emitted by a Sample, and the emitted volatile organic compounds was set at 100% for use in relation to the determination of volatile organic compounds emitted by the hot melt adhesive composition of Example 4. The volatile organic compound peak area was 716,723.

### Example 4

The composition of Example 4 was the hot melt adhesive composition of Control 1 and 5% by weight MOLSIV 13X adsorbent particles. The composition of Example 4 exhibited a viscosity of 13,450 cP of at 163° C.

The composition of Example 4 was tested according to the Test Method for Determining the Relative Amount of Volatiles Emitted by a Sample and the volatile organic compound peak area was determined to be 159,717 and the reduction in emitted volatile organic compounds relative to Control 1 was 77%.

Other embodiments are within the claims. For example, in one embodiment the composition includes a carrier (e.g., from about 50% by weight to 90% by weight carrier) and from about 10% by weight to no greater than about 50% by weight volatile organic compound sorbent particles. Useful carriers include waxes, petroleum jelly, greases, oils (e.g., low molecular weight polyisobutylene oils), and combinations thereof.

Suitable waxes include, e.g., functionalized waxes, non-functionalized waxes, and combinations thereof, including, e.g., Fischer Tropsch waxes (e.g., oxidized Fischer-Tropsch waxes), polyolefin waxes (e.g., polypropylene waxes, polyethylene waxes, high density low molecular weight polyethylene waxes, and by-product polyethylene waxes), paraffin waxes, microcrystalline waxes, metallocene waxes, vegetable waxes, animal waxes, stearamide waxes, glycerin monostearate, sorbitan monostearate, and combinations thereof. Suitable greases include, e.g., soaps (e.g., calcium stearate, sodium stearate, lithium stearate and combinations thereof) emulsified with oil (e.g., mineral oil, vegetable oil, and combinations thereof).

In another embodiment, the composition is an elastic composition that includes an elastic material and sorbent particles. The composition can be in the form of an elastic article and can include the sorbent particles at a variety of locations including, e.g., on the elastic material, in the elastic material, and combinations thereof. Elastic materials, when deformed, exhibit internal resistance that restrains the deformation and returns the material to its original state as the strain is reduced or removed. Elastic materials can have some loss of recovery that is non-reversible, which loss of recovery will depend on the distance and duration over which the material has been subject to strain. Examples of useful elastic materials include polyethylene elastomers, polypropylene elastomers, polyester elastomers, ethylene-propylene-diene terpolymers, styrene-isoprene-styrene, styrene-butadiene-styrene, styrene-ethylene/butylene-styrene, styrene-ethylene/propylene-styrene, polyurethane, polyisoprene, cross-linked polybutadiene, natural rubber, and combinations thereof. The elastic article preferably includes from about 0.1% by weight to about 10% by weight, at least about 0.5% by weight, no greater than about 10% by weight, no greater than about 5% by weight, no greater than about 3% by weight, no greater than about 2.5% by weight, no greater than about 2% by weight, no greater than about 1.5% by weight, from about 0.1% by weight to about 7.5% by weight, or even from about 0.1 to about 6% by weight volatile organic compound sorbent particles.

1. A hot melt adhesive composition including:
from about 5% by weight to about 80% by weight thermoplastic polymer;
from about 20% by weight to about 70% by weight tackifying agent; and
from about 0.5% by weight to about 10% by weight volatile organic compound sorbent particles,
the composition exhibiting a viscosity of no greater than 15,000 centipoise at 177° C.

2. The hot melt adhesive composition of paragraph 1, wherein the composition is sprayable.

3. The hot melt adhesive composition any of paragraphs 1-2, wherein the composition emits at least 20% less volatiles relative to the composition without of the volatile organic compound sorbent particles.

4. The hot melt adhesive composition of any of paragraphs 1-3, wherein the composition emits at least 50% less volatiles relative to the composition without of the volatile organic compound sorbent particles.

5. The hot melt adhesive composition of any of paragraphs 1-4, wherein the composition is free of visible settling after aging for 24 hours at 163° C.

6. The hot melt adhesive composition of any of paragraphs 1-5, wherein the composition is free of visible settling after aging for 72 hours at 163° C.

7. The hot melt adhesive composition of any of paragraphs 1-6 further including from about 5% by weight to about 35% by weight plasticizer.

8. The hot melt adhesive composition of any of paragraphs 1-7 including from about 0.5% by weight to about 5% by weight of the volatile organic compound sorbent particles.

9. The hot melt adhesive composition of any of paragraphs 1-9 wherein the sorbent particles comprise at least one of zeolite, alkali metal alumino-silicate, silica gel, silica-magnesia gel, silica-alumina gel, activated carbon, activated alumina, calcium oxide, and cyclodextrin.

10. The hot melt adhesive composition of any of paragraphs 1-9, wherein the thermoplastic polymer includes a styrene block copolymer.

11. The hot melt adhesive composition of any of paragraphs 1-10, wherein the thermoplastic polymer includes a polyolefin including at least one of an amorphous polyalphaolefin and a metallocene catalyzed polyolefin.

12. The hot melt adhesive composition of any of paragraphs 1-11 including:
from about 5% by weight to about 50% by weight thermoplastic polymer including at least one of block copolymer and polyolefin;
from about 30% by weight to about 65% by weight tackifying agent; and
from about 5% by weight to about 35% by weight plasticizer.

13. The hot melt adhesive composition of any of paragraphs 1-12 further including from about 0.5% by weight to about 5% by weight of the volatile organic compound sorbent particles.

14. The hot melt adhesive composition of any of paragraphs 1-13, wherein the composition is a pressure-sensitive hot melt adhesive composition.

15. A thermoplastic composition including:
from about 5% by weight to about 90% by weight a first component selected from the group consisting of thermoplastic polymer, tackifying agent and combinations thereof; and
from about 5% by weight to no greater than about 70% by weight volatile organic compound sorbent particles,
the composition exhibiting a viscosity no greater than 40,000 centipoise at 177° C.

16. The thermoplastic composition of paragraph 15 further including from about 5% by weight to no greater than about 50% by weight plasticizer;

17. The thermoplastic composition of any of paragraphs 15-16, wherein the composition exhibits a viscosity no greater than 15,000 centipoise at 177 ° C.

18. The thermoplastic composition of any of paragraphs 15-17, wherein the composition is sprayable.

19. The thermoplastic composition of any of paragraphs 15-18, wherein the first component includes a thermoplastic polymer and a tackifying agent.

20. An elastic attachment adhesive composition including:
from about 10% by weight to about 80% by weight thermoplastic elastomer;
from about 20% by weight to about 70% by weight tackifying agent; and
from about 0.1% by weight to about 10% by weight volatile organic compound sorbent particles,
the composition exhibiting a viscosity of no greater than 15,000 centipoise at 177° C.

21. The thermoplastic elastic attachment adhesive composition of paragraph 20 further including an end block resin.

22. A disposable absorbent article including:
an absorbent core; and
the composition of any of paragraphs 1-21.

24. A method of reducing an odor from a package, the odor being due to volatile organic compounds, the method including:
surrounding an article that includes the hot melt adhesive composition of any of paragraphs 1-14 or the thermoplastic composition of any of paragraphs 15-21 with a packaging material including a flexible film to form the package, at least one of the article and the packaging material emitting volatile organic compounds in the absence of volatile organic compound sorbent particles.

25. A method of reducing an odor that is perceived to be present when a package is opened, the method including:
surrounding an article including the hot melt adhesive composition of any of paragraphs 1-14 or the thermoplastic composition of any of paragraphs 15-21 with a packaging material to form the package, at least one of the article and the packaging material emitting volatile organic compounds; and
allowing the volatile organic compound sorbent particles to adsorb at least some of the volatile organic compounds.

26. A method of reducing an odor that is perceived to be present when a package that includes an article surrounded by a packaging material is opened, the method including:
adding a thermoplastic composition including a thermoplastic polymer and volatile organic compound sorbent particles to at least one of the article and the packaging material; and
enclosing the article within the packaging material, the packaging material including a flexible polymer film.

27. A method of reducing the volatile organic compounds emitted by a hot melt adhesive composition, the method including:
combining from about 0.5% by weight to about 10% by weight volatile organic compound sorbent particles, from about 5% by weight to about 80% by weight thermoplastic polymer, from about 20% by weight to about 70% by weight tackifying agent; and plasticizer to form a hot melt adhesive composition,
the hot melt adhesive composition exhibiting a viscosity of no greater than 15,000 centipoise at 177° C.

28. The method of paragraph 27, wherein the composition emits at least 20% less organic volatile compounds relative to the composition without the volatile organic compound sorbent particles.

29. The method of paragraph 24, wherein the package is an airtight, sealed package.

30. An organic volatile compound sorbent composition including:
a carrier including at least one of wax, petroleum jelly, grease, oil; and
from about 0.5% by weight to about 10% by weight volatile organic compound sorbent particles disposed in the carrier.

31. The sorbent composition of paragraph 30, wherein the composition exhibits a viscosity no greater than 15,000 centipoise at 177° C.

32. An elastic article including:
an elastic component; and
from about 0.5% by weight to about 10% by weight volatile organic compound sorbent particles, the volatile organic compound sorbent particles being at least one of disposed in the elastic component and disposed on the elastic component.

What is claimed is:

1. A hot melt adhesive composition comprising:
from about 5% by weight to about 80% by weight thermoplastic polymer;
from about 20% by weight to about 70% by weight tackifying agent; and
from about 0.5% by weight to about 10% by weight volatile organic compound sorbent particles,
the composition exhibiting a viscosity of no greater than 15,000 centipoise at 177° C.

2. The hot melt adhesive composition of claim 1, wherein the composition is sprayable.

3. The hot melt adhesive composition of claim 1, wherein the composition emits at least 20% less volatiles relative to the composition without of the volatile organic compound sorbent particles.

4. The hot melt adhesive composition of claim 1, wherein the composition is free of visible settling after aging for 24 hours at 163° C.

5. The hot melt adhesive composition of claim 1 further comprising from about 5% by weight to about 35% by weight plasticizer.

6. The hot melt adhesive composition of claim 1 comprising from about 0.5% by weight to about 5% by weight of the volatile organic compound sorbent particles.

7. The hot melt adhesive composition of claim 1 wherein the sorbent particles comprise zeolite, alkali metal alumino-silicate, silica gel, silica-magnesia gel, silica-alumina gel, activated carbon, activated alumina, calcium oxide, cyclodextrin, or a combination thereof.

8. The hot melt adhesive composition of claim 1, wherein the thermoplastic polymer comprises a styrene block copolymer, an amorphous polyalphaolefin, a metallocene catalyzed polyolefin, or a combination thereof.

9. The hot melt adhesive composition of claim 1 comprising
from about 5% by weight to about 50% by weight thermoplastic polymer comprising a block copolymer, a polyolefin, or a combination thereof;
from about 30% by weight to about 65% by weight tackifying agent; and
from about 5% by weight to about 35% by weight plasticizer.

10. The hot melt adhesive composition of claim 9 further comprising from about 0.5% by weight to about 5% by weight of the volatile organic compound sorbent particles.

11. The hot melt adhesive composition of claim 1, wherein the composition is a pressure-sensitive hot melt adhesive composition.

12. A thermoplastic composition comprising:
from about 5% by weight to about 90% by weight a first component selected from the group consisting of thermoplastic polymer, tackifying agent and combinations thereof; and
from about 5% by weight to no greater than about 70% by weight volatile organic compound sorbent particles,
the composition exhibiting a viscosity no greater than 40,000 centipoise at 177° C.

13. The thermoplastic composition of claim 12 further comprising from about 5% by weight to no greater than about 50% by weight plasticizer.

14. The thermoplastic composition of claim 12, wherein the composition exhibits a viscosity no greater than 15,000 centipoise at 177° C.

15. The thermoplastic composition of claim 12, wherein the composition is sprayable.

16. The thermoplastic composition of claim 12, wherein the first component comprises thermoplastic polymer and tackifying agent.

17. An elastic attachment adhesive composition comprising:
from about 10% by weight to about 80% by weight thermoplastic elastomer;
from about 20% by weight to about 70% by weight tackifying agent; and
from about 0.1% by weight to about 10% by weight volatile organic compound sorbent particles,
the composition exhibiting a viscosity of no greater than 15,000 centipoise at 177° C.

18. The thermoplastic elastic attachment adhesive composition of claim 17 further comprising an end block resin.

19. A disposable absorbent article comprising:
an absorbent core; and
the composition of claim 1.

20. A method of reducing an odor from a package, the odor being due to volatile organic compounds, the method comprising:
surrounding an article comprising the hot melt adhesive composition of claim 1 with a packaging material comprising a flexible film to form the package,
a component selected from the group consisting of the article, the packaging material, and combinations thereof emitting an odor.
